(19) United States
(12) Patent Application Publication
(10) Pub. No.: US 2011/0237984 A1
(43) Pub. Date: Sep. 29, 2011

(54) DYNAMIC SYSTEM FOR SHOCKWAVE TREATMENT

(76) Inventor: Moshe Ein-Gal, Ramat Hasharon (IL)

(21) Appl. No.: 12/729,262

(22) Filed: Mar. 23, 2010

(51) Int. Cl. A61B 17/225; A61B 5/05

(52) U.S. Cl. 601/4; 600/407

(57) ABSTRACT

A dynamic system for shockwave treatment including a shockwave source operative to produce shockwaves that propagate along a shockwave axis, an imaging beam source attached to the shockwave source, operable to emit an imaging beam along a beam axis, the shockwave source and the imaging beam source forming an assembly, an imaging detector operative to receive the imaging beam and generate signals thereby for processing into an image, and a positioner coupled to the assembly operative to position the assembly at a desired position and attitude in three-dimensional space, wherein said shockwave source is movable along the shockwave axis relative to the imaging beam source.

### FIELD OF THE INVENTION

The present invention relates to generation and focusing of energy waves in general, e.g., acoustic waves, and particularly to a system for shockwave treatment with imaging, wherein the shockwave generator and imaging beam generator are rigidly fixed relative to one another.

### BACKGROUND OF THE INVENTION

Extracorporeal shockwave treatment (ESWT) is a treatment modality for a variety of applications including disintegration of urinary tract calculi, disintegration of any stone-like concretions or depositions of minerals and salts found in ducts, blood vessels or hollow organs of a patient's body, advancing bone union by causing micro-fractures and relieving pain associated with tendons, joints and bony structures.

One well-known example of ESWT is extracorporeal shockwave lithotripsy (ESWL), in which a lithotripter having a shockwave head coupled to a patient's body, delivers shockwave energy to disintegrate the calculi.

In prior art ESWL, the target is localized by triangulation. X-ray imaging apparatus is provided that can rotate with respect to the shockwave source. The x-ray imaging apparatus is set at first rotational setting so that the x-ray beam axis intersects the shockwave propagation axis at a first angle. The x-ray imaging apparatus is then rotated to a second rotational setting so that the x-ray beam axis intersects the shockwave propagation axis at a second angle, and so forth. In this manner, a plurality of images are obtained of the target from different orientations. At each orientation, a discrepancy may be present between projections of the target and the shockwave focus. The target can them be moved to the shockwave focus by moving the treatment couch, on which the patient lies, relative to the shockwave focus. When the target position coincides with that of the shockwave focus, the respective discrepancies are practically reduced to zero. The patient is generally horizontal and so is the rotational axis of the x-ray imager.

The prior art apparatus has limitations. Treatment is not possible for a seated patient; tracking a moving target (e.g., due to respiration) is not done since it would require constant patient motion; rotating the x-ray imager for triangulated localization prevents attaching an x-ray shield to the x-ray detector.

### SUMMARY OF THE INVENTION

The present invention seeks to provide a novel shockwave treatment system, as is described more in detail hereinbelow, which has use in many medical applications, such as but not limited to, extracorporeal shockwave treatment (ESWT). The invention also has non-medical applications, such as but not limited to, non-destructive testing of structures.

In one non-limiting embodiment of the invention, the shockwave treatment system includes a treatment couch that is generally stationary during treatment. Motion is applied to an assembly of a shockwave source and an imaging beam source rigidly immovable with respect to each other. Triangulated target localization is obtained by translating the assembly without rotation relative to the target and utilizing an imaging beam, e.g., a conical x-ray beam. Assembly motion may be used for shaping the shockwave focal volume and/or for tracking the target during respiration.

There is thus provided in accordance with an embodiment of the invention a dynamic system for shockwave treatment including a shockwave source operative to produce shockwaves that propagate along a shockwave axis, an imaging beam source attached to the shockwave source, operable to emit an imaging beam along a beam axis, the shockwave source and the imaging beam source forming an assembly, an imaging detector operative to receive the imaging beam and generate signals thereby for processing into an image, and a positioner coupled to the assembly operative to position the assembly at a desired position and attitude in three-dimensional space wherein the shockwave source is operable to move along the shockwave axis relative to the imaging beam source. Preferably, although not necessarily, the beam axis is collinear with the shockwave axis.

In accordance with an embodiment of the invention the positioner includes a translatory actuator that moves the assembly in translation and/or a rotary actuator that rotates the assembly.

In accordance with an embodiment of the invention a support surface supports a patient thereupon. The support surface may be stationary or movable through a variable elevation angle.

In accordance with an embodiment of the invention a motion controller is in communication with the positioner that controls operation of the positioner such that the assembly of the shockwave source and the imaging beam source is moved in accordance with a desired pattern. One or more sensors or fiduciary implants may be provided to sense target location relative to the assembly of the shockwave source and the imaging beam source.

### BRIEF DESCRIPTION OF THE DRAWINGS

The present invention will be understood and appreciated more fully from the following detailed description taken in conjunction with the drawings in which:

FIG. 1 is a simplified sectional illustration of a dynamic system for shockwave treatment, constructed and operative in accordance with an embodiment of the invention.

### DETAILED DESCRIPTION OF EMBODIMENTS

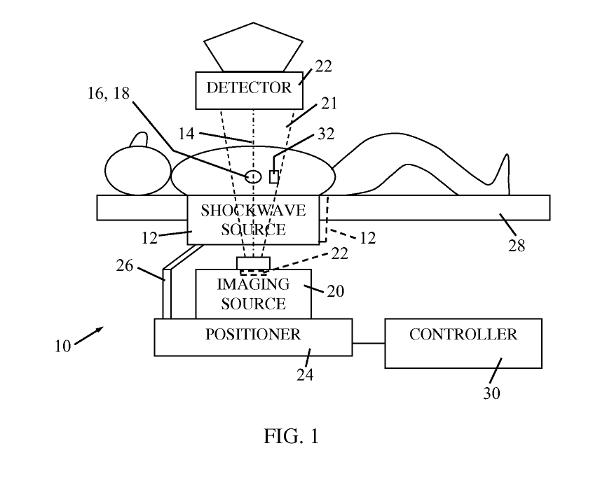

Reference is now made to FIG. 1, which illustrates a dynamic system 10 for shockwave treatment, constructed and operative in accordance with a non-limiting embodiment of the invention.

System 10 includes a shockwave source 12 that produces shockwaves 13 that propagate along a shockwave axis 14. Shockwave source 12 may include, without limitation, an electrical-to-shockwave energy converter (e.g., electro-hydraulic, electromagnetic or piezoelectric) and a focusing mechanism (e.g., shockwave lenses and/or ellipsoidal, parabolic or other shaped reflectors) for directing the shockwave energy to a focus 16, located at in a target 18 of a patient. The shockwave focusing mechanism may be cylindrically symmetric about the shockwave propagation axis 14.

An imaging beam source 20 is attached to a portion of shockwave source 12, and emits an imaging beam 21 along an imaging axis, preferably collinear with axis 14. Thus, axis 14 will also be referred to as the mutual beam axis 14, or simply the beam axis 14. Imaging beam source 20 may include, without limitation, an x-ray source or ultrasonic beam source. An imaging detector 22 is provided for receiving (capturing) the imaging beam and generating signals thereby for processing into an image, as is well known in the art. In the case of x-ray imaging, the imaging detector 22 is positioned to receive the beam after it has passed through the target area; in the case of ultrasonic imaging, the imaging detector 22 (shown optionally in broken lines in the drawing) is the ultrasonic transducer that receives the echoes of the ultrasonic beam reflected back through the target area.

Additional shockwave sources (shown optionally in broken lines) may be provided, which may operate synchronously or asynchronously. The additional sources may be collinear with the imaging beam or offset therefrom.

The assembly of shockwave source 12 and imaging beam source 20 is coupled to (mounted on or connected to) a positioner 24, operative to position the assembly at a desired position and attitude in three-dimensional space. Positioner 24 may, for example, be an X-Y-Z or just X-Y translatory actuator that moves the assembly in translation in one, two or three orthogonal axes of motion. Positioner 24 may, for example, be a rotary actuator that rotates the assembly about one two or three orthogonal axes of rotation (elevation, azimuth and roll). Of course, positioner 24 may be a combination of both for moving the assembly in translation and rotation.

Shockwave source 12 may be directly attached to imaging beam source 20 by suitable mechanical fasteners. Alternatively, shockwave source 12 may be rigidly attached to positioner 24 (e.g., with a mounting bracket 26) and imaging beam source 20 may be rigidly attached to positioner 24 (e.g., with fasteners), so that shockwave source 12 and imaging beam source 20 are in all embodiments rigidly fixed relative to each other.

In one embodiment of the invention, shockwave source 12 is rigidly attached to imaging beam source 20. In another embodiment of the invention, shockwave source 12 is not rigidly attached to imaging beam source 20, so that positioner 24 causes independent translation of shockwave source 12 without translating imaging beam source 20. Accordingly, positioner 24 can translate the sources together and/or separately (rather than the sources being rigidly attached).

The patient is supported on a support surface 28 (table, couch or seat). In the illustration, the patient is in a horizontal supine position. However, one of the advantages of positioner 24 being capable of rotating the assembly is the positioner 24 can vary the elevation angle of the assembly with respect to support surface 28. This enables treatment of the patient in any position, e.g., sitting, reclining or lying down. The support surface 28 is preferably stationary, but can be at least partially movable. For example, support surface 28 can be raised or lowered through a variable elevation angle so as to match, for example, the elevation angle of the assembly and allow treatment of a sitting, reclining or lying down patient.

Positioner 24 may be used to translate the assembly of shockwave source 12 and imaging beam source 20 such that images of the target 18 may be obtained from two different angles, thereby enabling target localization via triangulation. As is well known, in triangulation, the coordinates and distance to the target can be found by calculating the length of one side of a triangle, given measurements of angles and sides of the triangle formed by the target and two other known reference points.

In accordance with an embodiment of the invention, a motion controller 30 is in communication with positioner 24. The controller 30 controls operation of positioner 24 such that the assembly of shockwave source 12 and imaging beam source 20 is moved in accordance with a desired pattern for a desired treatment, diagnostic plan or imaging scheme. One or more sensors or fiduciary implants 32 may be used to sense target location relative to the assembly of shockwave source 12 and imaging beam source 20. The sensors or fiduciary implants 32 communicate with controller 30 and positioner 24 to effect a close-loop control of the position of the assembly.

It is appreciated that various features of the invention which are, for clarity, described in the contexts of separate embodiments, may also be provided in combination in a single embodiment. Conversely, various features of the invention which are, for brevity, described in the context of a single embodiment, may also be provided separately or in any suitable subcombination.

What is claimed is:

1. A dynamic system for shockwave treatment comprising:
a shockwave source operative to produce shockwaves that propagate along a shockwave axis;
an imaging beam source attached to said shockwave source, operable to emit an imaging beam along a beam axis, said shockwave source and said imaging beam source forming an assembly;
an imaging detector operative to receive the imaging beam and generate signals thereby for processing into an image; and
a positioner coupled to said assembly operative to position said assembly at a desired position and attitude in three-dimensional space, wherein said shockwave source is movable along the shockwave axis relative to the imaging beam source.

2. The system according to claim 1, wherein said beam axis is collinear with said shockwave axis.

3. The system according to claim 1, wherein said positioner comprises a translatory actuator that moves the assembly in translation.

4. The system according to claim 1, wherein said positioner comprises a rotary actuator that rotates the assembly.

5. The system according to claim 1, wherein said positioner comprises a combination of a translatory actuator that moves the assembly in translation and a rotary actuator that rotates the assembly.

6. The system according to claim 1, wherein said shockwave source comprises a plurality of shockwave sources.

7. The system according to claim 1, wherein said shockwave source is attached to said imaging beam source by mechanical fasteners.

8. The system according to claim 1, wherein said shockwave source and said imaging beam source are both rigidly attached to said positioner.

9. The system according to claim 1, further comprising a support surface for supporting a patient thereupon.

10. The system according to claim 9, wherein said support surface is stationary.

11. The system according to claim 9, wherein said support surface is movable through a variable elevation angle.

12. The system according to claim 1, further comprising a motion controller in communication with said positioner that controls operation of said positioner such that the assembly of said shockwave source and said imaging beam source is moved in accordance with a desired pattern.

13. The system according to claim 1, further comprising one or more sensors or fiduciary implants to sense target location relative to the assembly of said shockwave source and said imaging beam source.
